(19) United States
(12) Patent Application Publication
(10) Pub. No.: US 2022/0319694 A1
(43) Pub. Date: Oct. 6, 2022

(54) SYSTEMS AND METHODS FOR TELEMETRIC HEALTH MONITORING UTILIZING A DATA AND COMMUNICATIONS HUB

(71) Applicant: KoKo IT, LLC, Longmont, CO (US)

(72) Inventors: John Robert Peterson, Chardon, OH (US); Norman Thomas, Hopedale, MA (US)

(21) Appl. No.: 17/712,922

(22) Filed: Apr. 4, 2022

Related U.S. Application Data
(60) Provisional application No. 63/170,071, filed on Apr. 2, 2021.

(51) Int. Cl. G16H 40/67; G16H 10/60

(52) U.S. Cl. CPC G16H 40/67; G16H 10/60

(57) ABSTRACT

Embodiments of the present disclosure relate generally to network enabled devices and systems that allow a user to monitor various aspects of their physiological status through a cloud-based healthcare system that includes one or more sensor devices, a data and communications hub (e.g., embedded device), and a communication network based on remote server. More particularly, embodiments of the present disclosure comprise devices, systems, and methods that provide users with the ability to accurately assess their physiological function (e.g., pulmonary function) using a handheld sensor device (e.g., a spirometer) that is in communication with a data and communications hub configured for transmitting data to and receiving data from a cloud-based network on a remote server.

### CROSS REFERENCE TO RELATED APPLICATION

The present application claims the benefits of and priority, under 35 U.S.C. § 119(e), to U.S. Provisional Application No. 63/170,071 filed Apr. 2, 2021 by Peterson, et al and entitled “Systems and Methods for Telemetric Health Monitoring Utilizing a Central Data and Communications Hub” of which the entire disclosure is incorporated herein by reference for all purposes.

### FIELD OF THE DISCLOSURE

Embodiments of the present disclosure relate generally to methods and systems for enabling a user to monitor various aspects of their physiological status through a cloud-based healthcare system that includes one or more sensor devices, a data hub (e.g., an embedded device), and a communications network based on remote server and more particularly to devices, systems, and methods that provide users with the ability to accurately assess their physiological function (e.g., pulmonary function) using a handheld sensing device (e.g., a spirometer) that is in communication with a data hub configured for transmitting data to and receiving data from a cloud-based network on a remote server.

### BACKGROUND

Spirometry encompasses several common tests used for accessing a person's pulmonary health and respiratory capabilities in terms of lung function parameters relating to the amount (volume) and/or speed (flow, or flow rate) of air that can be inhaled (inspiratory) and exhaled (expiratory), either forcibly or under normal breathing. The primary signals measured in spirometry include inhalation volume and/or flow. Results are typically provided in the form of raw data such as measured liters and liters per second as well as percentages of predicted values in relation to predicted values for patients of similar parameters such as height, age, sex, weight, and the like. Results from spirometry testing are often displayed graphically, in so called spirograms or pneumotachographs that show a volume-time curve (volume in liters on the Y-axis and time in seconds on the X-axis) and/or a flow-volume loop (depicting the rate of airflow on the Y-axis and the total volume inhaled or exhaled on the X-axis).

Spirometry is an important tool in the assessment of various obstructive or restrictive lung conditions such as asthma, chronic obstructive pulmonary disease (“COPD”), bronchitis, emphysema, pulmonary fibrosis (“PF”), and also cystic fibrosis (“CF”), because the tests performed with the spirometer (a device, or apparatus, for measuring ventilation, the movement of air into and out of the lungs) are able to identify abnormal ventilation patterns, namely obstructive and restrictive patterns. Indeed, it is very important to be able to measure both inspiratory and expiratory volumes during spirometry to accurately diagnosis whether a patient has an obstructive or restrictive lung disease. Obstructive lung diseases include conditions that make it hard for the patient to exhale all the air in their lungs whereas in the case of restrictive lung disease patients have difficulty fully expanding their lungs with air. Obstructive and restrictive lung disease share the same main symptom: shortness of breath with exertion. In addition to the diagnostic value of spirometry, these tests also allow physicians to monitor whether a particular medicine or respiratory therapy is controlling or improving a patient's breathing. Spirometry is further used by clinicians to monitor chronic respiratory diseases and distress such as chronic obstructive pulmonary disease (“COPD”) and the like.

Various types of spirometers are known from simple mechanical devices to fully electronic devices, typical inspiratory and expiratory sensors include water gauges, windmill-type rotors (e.g., so-called turbines) and pressure transducers. In general, these devices consists of analog and digital spirometry devices. Most analog devices are only capable of measuring expiratory peak flow and FEV1. Analog devices are incapable of inspiratory measurements such as FVC and hence do not present a full clinical test. For this reason and others, the global market for analog spirometry devices has gradually been ceded to digital lung function testing devices. Digital spirometers fall broadly into two categories: connected devices (that connect to a smartphone, tablet, or Personal Computer (“PC”) and unconnected devices that have an LCD screen for user convenience.

However, there is a need for improved methods and systems for cost effective healthcare related sensors, such as the disclosed spirometers, and accompanying communications systems and monitoring methods that can initiate and receive communications from a central monitoring organization to provide the benefits of telemedicine to users in situations where it is not possible to employ expensive personal computers or smartphones as part of the communications chain.

### BRIEF SUMMARY

Embodiments of the disclosure provide systems and methods for monitoring telemetric health data. According to one embodiment, a server of a telemetric health monitoring system, the server can comprise a processor and a memory coupled with and readable by the processor. The memory can store therein a set of instructions which, when executed by the processor, causes the processor to receive set-up data for a telemetric health data sensing device and maintain the received set-up data for the telemetric health data sensing device in an electronic record corresponding to a user of the telemetric health data sensing device. For example, the set-up data for the telemetric health data sensing device can comprise one or more of a serial number for the telemetric health data sensing device, a date, or demographic information for the user of the telemetric health data sensing device.

The instructions can further cause the processor to determine whether to collect telemetric health data corresponding to the user of the telemetric health data sensing device. For example, determining whether to collect the telemetric health data corresponding to the user of the telemetric health data sensing device can be based at least in part on the set-up data for the telemetric health data sensing device.

In response to determining to collect the telemetric health data corresponding to the user of the telemetric health data sensing device, the instructions can further cause the processor to send an electronic message requesting the telemetric health data to a hub device communicatively coupled with the telemetric health data sensing device via a communications network, receive the requested telemetric health data from the hub device via the communications network, and update the electronic record corresponding to the user of the telemetric health data sensing device with the received telemetric health data. In some cases, the electronic message requesting the telemetric health data can comprise one or more of instructions to the hub device or instructions to the user of the telemetric health data sensing device. The requested telemetric health data can comprise, for example, one or more of a serial number for the telemetric health data sensing device, a time stamp for the requested telemetric health data, one or more data labels and numerical values for the requested telemetric health data, or questionnaire answers from the user of the telemetric health data sensing device.

In some cases, the instructions can further cause the processor to present a graphical user interface presenting at least a portion of the telemetric health data maintained in the electronic record corresponding to the user of the telemetric health data sensing device.

According to another embodiment, a data and communications hub device of a telemetric health monitoring system, the data and communications hub can comprise a processor and a memory coupled with and readable by the processor. The memory can store therein a set of instructions which, when executed by the processor, causes the processor to receive, from a server of the telemetric health monitoring system, via a communications network, an electronic message requesting telemetric health data corresponding to a user of a telemetric health data sensing device. In some cases, the electronic message requesting telemetric health data corresponding to the user of a telemetric health data sensing device further can comprise instructions to the user of the telemetric health data sensing device.

The set of instructions stored in the memory of the data and communications hub device can further cause the processor to collect data from the user of the telemetric health data sensing device, collect the telemetric health data from the telemetric health data sensing device, and provide the data collected from the user of the telemetric health data sensing device and the telemetric health data collected from the telemetric health data sensing device to the server via the communications network in response to the received electronic message. The telemetric health data collected from the telemetric health data sensing device can comprise one or more of a serial number for the telemetric health data sensing device, a time stamp for the telemetric health data, or one or more data labels and numerical values for the telemetric health data. In some cases, collecting data from the user of the telemetric health data sensing device further can comprise providing a wake up instruction to the telemetric health data sensing device. Additionally, or alternatively, collecting data from the user of the telemetric health data sensing device can comprise presenting a questionnaire to the user of the telemetric health data sensing device and wherein the data collected from the user of the telemetric health data sensing device comprises answers to the questionnaire.

According to yet another embodiment, a method for monitoring telemetric health data can comprise receiving, by a server of a telemetric health data monitoring system, set-up data for a telemetric health data sensing device, and maintaining, by the server of a telemetric health data monitoring system, the received set-up data for the telemetric health data sensing device in an electronic record corresponding to a user of the telemetric health data sensing device. The set-up data for the telemetric health data sensing device can comprise one or more of a serial number for the telemetric health data sensing device, a date, or demographic information for the user of the telemetric health data sensing device.

A determination can be made by the server of the telemetric health data monitoring system as to whether to collect telemetric health data corresponding to the user of the telemetric health data sensing device. Determining whether to collect the telemetric health data corresponding to the user of the telemetric health data sensing device can be based at least in part on the set-up data for the telemetric health data sensing device. In response to determining to collect the telemetric health data corresponding to the user of the telemetric health data sensing device, an electronic message requesting the telemetric health data can be sent by the server of the telemetric health data monitoring system to a hub device communicatively coupled with the telemetric health data sensing device via a communications network. The electronic message requesting the telemetric health data comprises one or more of instructions to the hub device or instructions to the user of the telemetric health data sensing device.

The hub device of the telemetric health data monitoring system can receive, from the server of the telemetric health monitoring system, via the communications network, the electronic message requesting telemetric health data corresponding to a user of a telemetric health data sensing device, collect data from the user of the telemetric health data sensing device and/or collect the telemetric health data from the telemetric health data sensing device. The requested telemetric health data can comprise, for example, one or more of a serial number for the telemetric health data sensing device, a time stamp for the requested telemetric health data, one or more data labels and numerical values for the requested telemetric health data, or questionnaire answers from the user of the telemetric health data sensing device. In some cases, collecting data from the user of the telemetric health data sensing device can further comprise waking up the telemetric health data sensing device. The data collected from the user of the telemetric health data sensing device and the telemetric health data collected from the telemetric health data sensing device can then be provided by the hub device of the telemetric health data monitoring system to the server via the communications network in response to the received electronic message.

The server of the telemetric health data monitoring system can receive the data collected from the user of the telemetric health data sensing device and the telemetric health data collected from the telemetric health data sensing device from the hub device via the communications network and update the electronic record corresponding to the user of the telemetric health data sensing device with the received the data collected from the user of the telemetric health data sensing device and the telemetric health data collected from the telemetric health data sensing device.

### BRIEF DESCRIPTION OF THE DRAWINGS

FIG. 1 is a block diagram illustrating elements of an exemplary computing environment in which embodiments of the present disclosure may be implemented.

FIG. 2 is a block diagram illustrating elements of an exemplary computing device in which embodiments of the present disclosure may be implemented.

FIG. 3A is a block diagram illustrating an exemplary environment in which embodiments of the present disclosure may be implemented.

FIG. 3B is a block diagram illustrating an alternative exemplary environment in which embodiments of the present disclosure may be implemented.

FIG. 4 is a block diagram illustrating an exemplary embodiment of various modules forming a user specific healthcare monitoring and alert applications, which can be provided as part of the network system of FIG. 3A and FIG. 3B.

FIGS. 5A and 5B are block diagrams of exemplary devices and components therein according to embodiments of the present disclosure.

FIG. 6 is a flowchart illustrating an exemplary process for telemetric health monitoring according to one embodiment of the present disclosure.

FIG. 7 is a flowchart illustrating an exemplary process for telemetric health monitoring according to another embodiment of the present disclosure.

In the appended figures, similar components and/or features may have the same reference label. Further, various components of the same type may be distinguished by following the reference label by a letter that distinguishes among the similar components. If only the first reference label is used in the specification, the description is applicable to any one of the similar components having the same first reference label irrespective of the second reference label.

### DETAILED DESCRIPTION

In the following description, for the purposes of explanation, numerous specific details are set forth in order to provide a thorough understanding of various embodiments disclosed herein. It will be apparent, however, to one skilled in the art that various embodiments of the present disclosure may be practiced without some of these specific details. The ensuing description provides exemplary embodiments only and is not intended to limit the scope or applicability of the disclosure. Furthermore, to avoid unnecessarily obscuring the present disclosure, the preceding description omits a number of known structures and devices. This omission is not to be construed as a limitation of the scopes of the claims. Rather, the ensuing description of the exemplary embodiments will provide those skilled in the art with an enabling description for implementing an exemplary embodiment. It should however be appreciated that the present disclosure may be practiced in a variety of ways beyond the specific detail set forth herein.

While the exemplary aspects, embodiments, and/or configurations illustrated herein show the various components of the system collocated, certain components of the system can be located remotely, at distant portions of a distributed network, such as a Local-Area Network (LAN) and/or Wide-Area Network (WAN) such as the Internet, or within a dedicated system. Thus, it should be appreciated, that the components of the system can be combined in to one or more devices or collocated on a particular node of a distributed network, such as an analog and/or digital telecommunications network, a packet-switch network, or a circuit-switched network. It will be appreciated from the following description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire and fiber optics, and may take the form of acoustic or light waves, such as those generated during radio-wave and infra-red data communications.

As used herein, the phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

The term “computer-readable medium” as used herein refers to any tangible storage and/or transmission medium that participate in providing instructions to a processor for execution. Such a medium may take many forms, including but not limited to, non-volatile media, volatile media, and transmission media. Non-volatile media includes, for example, Non-Volatile Random-Access Memory (NVRAM), or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a Compact Disk Read-Only Memory (CD-ROM), any other optical medium, punch cards, paper tape, any other physical medium with patterns of holes, a Random-Access Memory (RAM), a Programmable Read-Only Memory (PROM), and Erasable Programmable Read-Only Memory (EPROM), a Flash-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored.

A “computer readable signal” medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable medium may be transmitted using any appropriate medium, including but not limited to wireless, wireline, optical fiber cable, Radio Frequency (RF), etc., or any suitable combination of the foregoing.

The terms “determine,” “calculate,” and “compute,” and variations thereof, as used herein, are used interchangeably and include any type of methodology, process, mathematical operation or technique.

The terms “first,” “second,” and “third,” and so forth are used merely as labels, and are not intended to impose numerical requirements on their objects and may be used to simply distinguish a corresponding component from another, and do not limit the components in other aspect (e.g., importance or order).

Also, terms such as “element,” and “component,” encompass both elements and components comprising one unit and elements and components that comprise more than one subunit unless specifically stated otherwise. It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly or via the other element (e.g., a third element).

As used herein, the terms “device” and “test device” are used to mean any implement configured to obtain health related data about or from the user of the “device.” Health related data may include, but is not limited to, any one or more values related to the user's respiratory health and function. A “device” may comprise or provide the functionality of a sensor (e.g., a spirometer).

Recitation of ranges of values herein are merely intended to serve as a shorthand method of referring individually to each separate value falling within the range, unless otherwise indicated herein, and each separate value within the range is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to any one or more embodiments of the present disclosure.

Unless otherwise defined herein, technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition.

As used herein, the term “telemedicine” is used mean the uni-/bidirectional flow of information from a user/patient to a remote clinician (e.g., doctor, nurse, nurse practitioner, paramedic (first responder(s)), therapist, pharmacist, and the like), a remote computer implemented database, or a remote computer implemented artificial intelligence system. Aspects within the use of term telemedicine as used herein include, but are not limited to, a user obtaining desired physiological information from one or more sensor devices and transmitting the information to a remote party, a user receiving questions posed by a remote party (e.g., clinician, database, artificial intelligence system, and the like) and transmitting their answers or other information back to the remote party (and the reverse), and a remote party (e.g., clinician, database, artificial intelligence system, and the like) transmitting instructions or other information to a user (and the reverse). The “information” transferred (i.e., sent and received) during telemedicine is not intended to be limited other than being perceptible with or without the aid of a computer processor, display, microphone, speaker, keyboard, mouse, digital pen, touchpad, printer, memory device, and the like.

The following terms, as used herein, as they are related to standard spirometric values are defined as follows:

Forced Vital Capacity (FVC) refers to the total volume of air that can be exhaled during a maximal forced expiration effort;

Forced Expiratory Volumen (FEVn) refers to the volume of air exhaled in the first specified “n” measure of time, typically measured in seconds, under force after a maximal inhalation. Thus, for example, FEV1 indicates the volume of air exhaled in the first one second under force after a maximal inhalation, likewise, FEV6 indicates the volume of air exhaled in the first six (6) seconds;

FEVn/FVC ratio refers to the percentage of the FVCn expired in the specified “n” measure of time, typically measured in seconds; thus, for example, an FEV1/FVC ratio indicates the percentage of the FVC1 expired in one second;

FEF25-75% refers to the Forced Expiratory Flow over the middle one half of the FVC that is the average flow from the point at which 25% of the FVC has been exhaled to the point at which 75% of the FVC has been exhaled; and

Maximal Voluntary Ventilation (MVV), also referred to as Maximal Breathing Capacity (MBC), describes the maximum minute volume of ventilation that the subject can maintain for 12 to 15 seconds.

The following terms, as used herein, as they are related to standard lung volumes are defined as follows:

Expiratory Reserve Volume (ERV) refers to the maximal volume of air exhaled from end expiration;

Inspiratory Reserve Volume (IRV) refers to the maximal volume of air inhaled from end inspiration;

Residual Volume (RV) refers to the volume of air remaining in the lungs after a maximal exhalation; and

Tidal Volume (VT) refers to the volume of air inhaled or exhaled during each respiratory cycle.

The following terms, as used herein, as they are related to lung capacities are defined as follows:

Functional Residual Capacity (FRC) refers to the volume of air in the lungs at resting end expiration;

Inspiratory Capacity (IC) refers to the maximal volume of air that can be inhaled from the resting expiratory level;

Total Lung Capacity (TLC) refers to the volume of air in the lungs at maximal inflation; and

Vital Capacity (VC) refers to the largest volume measured on complete exhalation after full.

As used herein, the term “test capability” is understood to mean, as applicable related to the specifically configured devices (e.g., sensors, data and communications hub (e.g., an embedded device), and the like) and systems of the present disclosure to be capable of acquiring and interpreting certain spirometric values, including, but not limited to: FEV1, FVC, FEV1/FVC, PEF, FET, FEV3, FEV3/FVC, FEV0.5, FEV0.75, FEV0.5/FVC, FEV0.75/FVC, FEV6, FEV1/FEV6, FEF25, FEF50, FEF75, MMEF25-50, MMEF25-75, PEFT, FEV1/FIVC, FIF25, FIF50, FIF75, FIV1, FIV1/FIVC, FIVC, PIF, ELA, EOTV0.5, EOTV1, EV, VC, IVC, IC, ERV, IRV, TV, TI, TE, tE/tTOT, VE, RR, TV/TI, TE/TI, and MVV, and the like.

It shall be understood that the term “means” as used herein shall be given its broadest possible interpretation in accordance with 35 U.S.C., Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary of the disclosure, brief description of the drawings, detailed description, abstract, and claims themselves.

Aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Any combination of one or more computer readable medium(s) may be utilized. The computer readable medium may be a computer readable signal medium or a computer readable storage medium.

In yet another embodiment, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as Programmable Logic Device (PLD), Programmable Logic Array (PLA), Field Programmable Gate Array (FPGA), Programmable Array Logic (PAL), special purpose computer, any comparable means, or the like. In general, any device(s) or means capable of implementing the methodology illustrated herein can be used to implement the various aspects of this disclosure. Exemplary hardware that can be used for the disclosed embodiments, configurations, and aspects includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

Examples of the processors as described herein may include, but are not limited to, at least one of Qualcomm® Snapdragon® 800 and 801, Qualcomm® Snapdragon® 610 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, Texas Instruments® Jacinto C6000™ automotive infotainment processors, Texas Instruments® OMAP™ automotive-grade mobile processors, ARM® Cortex™-M processors, ARM® Cortex-A and ARM926EJ-S™ processors, other industry-equivalent processors, and may perform computational functions using any known or future-developed standard, instruction set, libraries, and/or architecture.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or Very Large-Scale Integration (VLSI) design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or Common Gateway Interface (CGI) script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the aspects, embodiments, and/or configurations with reference to particular standards and protocols, the aspects, embodiments, and/or configurations are not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

Various additional details of embodiments of the present disclosure will be described below with reference to the figures. While the flowcharts will be discussed and illustrated in relation to a particular sequence of events, it should be appreciated that changes, additions, and omissions to this sequence can occur without materially affecting the operation of the disclosed embodiments, configuration, and aspects.

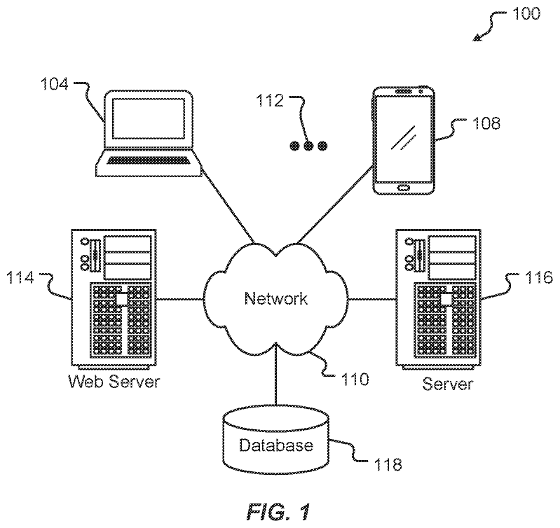

FIG. 1 is a block diagram illustrating elements of an exemplary computing environment in which embodiments of the present disclosure may be implemented. More specifically, this example illustrates a computing environment 100 that may function as the servers, user computers, or other systems provided and described herein. The environment 100 includes one or more user computers, or computing devices, such as a computing device 104, a communication device 108, and/or more 112. The computing devices 104, 108, 112 may include general purpose personal computers (including, merely by way of example, personal computers, and/or laptop computers running various versions of Microsoft Corp.'s Windows® and/or Apple Corp.'s Macintosh® operating systems) and/or workstation computers running any of a variety of commercially-available UNIX® or UNIX-like operating systems. These computing devices 104, 108, 112 may also have any of a variety of applications, including for example, database client and/or server applications, and web browser applications. Alternatively, the computing devices 104, 108, 112 may be any other electronic device, such as a thin-client computer, Internet-enabled mobile telephone, and/or personal digital assistant, capable of communicating via a network 110 and/or displaying and navigating web pages or other types of electronic documents. Although the exemplary computer environment 100 is shown with two computing devices, any number of user computers or computing devices may be supported.

Environment 100 further includes a network 110. The network 110 may can be any type of network familiar to those skilled in the art that can support data communications using any of a variety of commercially-available protocols, including without limitation Session Initiation Protocol (SIP), Transmission Control Protocol/Internet Protocol (TCP/IP), Systems Network Architecture (SNA), Internetwork Packet Exchange (IPX), AppleTalk, and the like. Merely by way of example, the network 110 maybe a Local Area Network (LAN), such as an Ethernet network, a Token-Ring network and/or the like; a wide-area network; a virtual network, including without limitation a Virtual Private Network (VPN); the Internet; an intranet; an extranet; a Public Switched Telephone Network (PSTN); an infra-red network; a wireless network (e.g., a network operating under any of the IEEE 802.9 suite of protocols, the Bluetooth® protocol known in the art, and/or any other wireless protocol); and/or any combination of these and/or other networks.

The system may also include one or more servers 114, 116. In this example, server 114 is shown as a web server and server 116 is shown as an application server. The web server 114, which may be used to process requests for web pages or other electronic documents from computing devices 104, 108, 112. The web server 114 can be running an operating system including any of those discussed above, as well as any commercially-available server operating systems. The web server 114 can also run a variety of server applications, including SIP servers, HyperText Transfer Protocol (secure) (HTTP(s)) servers, FTP servers, CGI servers, database servers, Java servers, and the like. In some instances, the web server 114 may publish operations available operations as one or more web services.

The environment 100 may also include one or more file and or/application servers 116, which can, in addition to an operating system, include one or more applications accessible by a client running on one or more of the computing devices 104, 108, 112. The server(s) 116 and/or 114 may be one or more general purpose computers capable of executing programs or scripts in response to the computing devices 104, 108, 112. As one example, the server 116, 114 may execute one or more web applications. The web application may be implemented as one or more scripts or programs written in any programming language, such as Java™, C, C#®, or C++, and/or any scripting language, such as Perl, Python, or Tool Command Language (TCL), as well as combinations of any programming/scripting languages. The application server(s) 116 may also include database servers, including without limitation those commercially available from Oracle®, Microsoft®, Sybase®, IBM® and the like, which can process requests from database clients running on a computing device 104, 108, 112.

The web pages created by the server 114 and/or 116 may be forwarded to a computing device 104, 108, 112 via a web (file) server 114, 116. Similarly, the web server 114 may be able to receive web page requests, web services invocations, and/or input data from a computing device 104, 108, 112 (e.g., a user computer, etc.) and can forward the web page requests and/or input data to the web (application) server 116. In further embodiments, the server 116 may function as a file server. Although for ease of description, FIG. 1 illustrates a separate web server 114 and file/application server 116, those skilled in the art will recognize that the functions described with respect to servers 114, 116 may be performed by a single server and/or a plurality of specialized servers, depending on implementation-specific needs and parameters. The computer systems 104, 108, 112, web (file) server 114 and/or web (application) server 116 may function as the system, devices, or components described herein.

The environment 100 may also include a database 118. The database 118 may reside in a variety of locations. By way of example, database 118 may reside on a storage medium local to (and/or resident in) one or more of the computers 104, 108, 112, 114, 116. Alternatively, it may be remote from any or all of the computers 104, 108, 112, 114, 116, and in communication (e.g., via the network 110) with one or more of these. The database 118 may reside in a Storage-Area Network (SAN) familiar to those skilled in the art. Similarly, any necessary files for performing the functions attributed to the computers 104, 108, 112, 114, 116 may be stored locally on the respective computer and/or remotely, as appropriate. The database 118 may be a relational database, such as Oracle 20i®, that is adapted to store, update, and retrieve data in response to Structured Query Language (SQL) formatted commands.

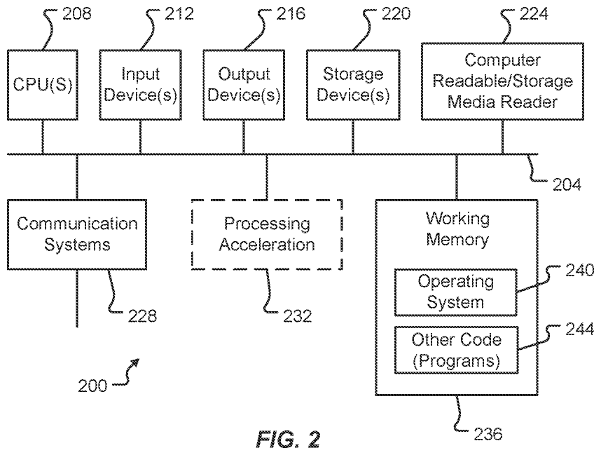

FIG. 2 is a block diagram illustrating elements of an exemplary computing device in which embodiments of the present disclosure may be implemented. More specifically, this example illustrates one embodiment of a computer system 200 upon which the servers, user computers, computing devices, or other systems or components described above may be deployed or executed. The computer system 200 is shown comprising hardware elements that may be electrically coupled via a bus 204. The hardware elements may include one or more Central Processing Units (CPUs) 208; one or more input devices 212 (e.g., a mouse, a keyboard, etc.); and one or more output devices 216 (e.g., a display device, a printer, etc.). The computer system 200 may also include one or more storage devices 220. By way of example, storage device(s) 220 may be disk drives, optical storage devices, solid-state storage devices such as a Random-Access Memory (RAM) and/or a Read-Only Memory (ROM), which can be programmable, flash-updateable and/or the like.

The computer system 200 may additionally include a computer-readable storage media reader 224; a communications system 228 (e.g., a modem, a network card (wireless or wired), an infra-red communication device, etc.); and working memory 236, which may include RAM and ROM devices as described above. The computer system 200 may also include a processing acceleration unit 232, which can include a Digital Signal Processor (DSP), a special-purpose processor, and/or the like.

The computer-readable storage media reader 224 can further be connected to a computer-readable storage medium, together (and, optionally, in combination with storage device(s) 220) comprehensively representing remote, local, fixed, and/or removable storage devices plus storage media for temporarily and/or more permanently containing computer-readable information. The communications system 228 may permit data to be exchanged with a network and/or any other computer described above with respect to the computer environments described herein. Moreover, as disclosed herein, the term “storage medium” may represent one or more devices for storing data, including ROM, RAM, magnetic RAM, core memory, magnetic disk storage mediums, optical storage mediums, flash memory devices and/or other machine-readable mediums for storing information.

The computer system 200 may also comprise software elements, shown as being currently located within a working memory 236, including an operating system 240 and/or other code 244. It should be appreciated that alternate embodiments of a computer system 200 may have numerous variations from that described above. For example, customized hardware might also be used and/or particular elements might be implemented in hardware, software (including portable software, such as applets), or both. Further, connection to other computing devices such as network input/output devices may be employed.

Examples of the processors 208 as described herein may include, but are not limited to, at least one of Qualcomm® Snapdragon® 800 and 801, Qualcomm® Snapdragon® 620 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, Texas Instruments® Jacinto C6000™ automotive infotainment processors, Texas Instruments® OMAP™ automotive-grade mobile processors, ARM® Cortex™-M processors, ARM® Cortex-A and ARM926EJ-S™ processors, other industry-equivalent processors, and may perform computational functions using any known or future-developed standard, instruction set, libraries, and/or architecture.

Embodiments of the present disclosure relate generally to network enabled devices and systems that allow a user to monitor various aspects of their physiological status through a cloud-based healthcare system that includes one or more sensor devices, a data and communications hub (e.g., embedded device), and a communication network based on remote server. More particularly, embodiments of the present disclosure comprise devices, systems, and methods that provide users with the ability to accurately assess their physiological function (e.g., pulmonary function) using a handheld sensor device (e.g., a spirometer) that is in communication with a data and communications hub configured for transmitting data to and receiving data from a cloud-based network on a remote server.

In various embodiments the devices and systems of the present disclosure operate with or without the use of a separate personal computer (e.g., laptop or desktop) and/or separate mobile device such as a smartphone that combines cellular and mobile computing functions. These embodiments being configured to comprises devices and systems being operable through communications links to and from a configurable communications hub unit that optionally comprises suitable wireless network protocols (e.g., based on IEEE 802/802.11 standards, including, but not limited to, WiFi and Wi-Fi Protected Access “WPA”), digital mobile networks (e.g., Global System for Mobile Communications “GSM,” Code Division Multiple Access “CDMA,” and the like), Global Position System components (e.g., GPS receives), Subscriber Identity Module (e., “SIM,” “SIM card”), and/or wired connections (e.g., USB 2.0, 3.0, 3.1, 3.2, 4; IEEE1394; Ethernet; MIDI; eSATA/eSATAp; Thunderbolt, 2, 3; and the like) to the sensing devices (e.g., a spirometer).

In one embodiment of the disclosure the system encompasses providing a device (e.g., an electronic respirometer) for obtaining and recording data related to at least one aspect of the user's respiration such as, but not limited to, tidal values and flow. In this sense, a respirometer device may be used. One or more of devices including, but not limited to, spirometers, incentive spirometers, pneumotachographs, peak flow meters, impedance pneumographs, or plethysmographs (e.g., photoplethysmographs, optoelectronic plethysmographs, respiratory inductance plethysmographs, and accelerometers) can be used to obtain and record user data.

In some embodiments, the spirometer device may identify a medical condition using the flow measurement results and for warning the user either directly, through processing and communication to and from the data and communications hub, or through processing and communication to and from a cloud-based communications network on a remote server. The spirometer device can further comprise input devices (e.g., keypad, tactile tracking device, touchscreen, microphone, and the like) to accommodate user entered information, which information may be used for enumerating and/or identifying one or more medical conditions. In alternative embodiments, the device may be in local communication with a mobile device, such as a cellphone or tablet, through which input and/or output may be provided. User entered information can be used for enumerating and/or identifying one or more medical conditions. Such information can be used, for example, to suggest preventive measures to the user upon identifying the medical condition.

According to one embodiment, the spirometer device can accommodate a dispenser with medicine for inhaling wherein the device can have a channel for delivery of the medication to the user's airways. Such medication can comprise, for example, a short acting and/or long acting bronchodilator.

Spirometer devices, and other contemplated sensors devices, can comprise storage (e.g., memory) for recording measurement data and a communication device such as IR port or radiofrequency device (e.g., BLUETOOTH®) and/or a wired communication (e.g., USB) means for data exchange with a data and communications hub, and optionally, or additionally, for communication with a PC. Communications can be unidirectional, e.g., from the sensor device to the communications hub or central hub, or bidirectional. The measurement data can be transferred over the internet and can be used in practice of telemedicine. In some embodiments, the communication enabled devices and components of the present disclosure can include interfaces (wired or wireless) to access a cellular phone network.

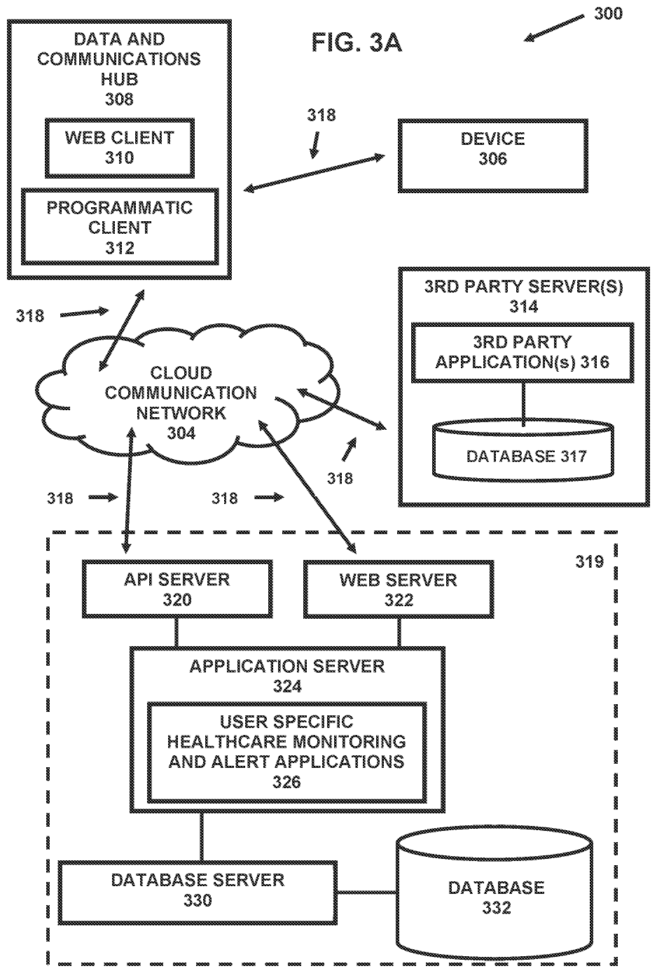

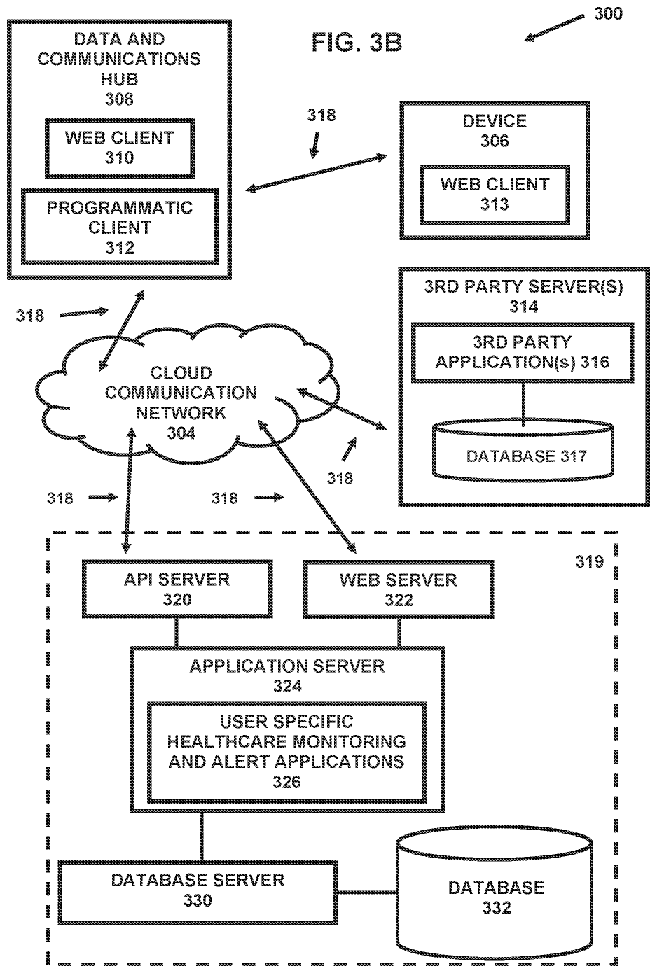

FIG. 3A is a block diagram illustrating an exemplary environment in which embodiments of the present disclosure may be implemented. More specifically, FIG. 3A provides an illustration of an exemplary network system 300 according to one embodiment having a client-server architecture configured for exchanging data over a network. The system 300 can be implemented using any number of servers, communications networks, computing devices, etc. as described above. In FIG. 3A the network system 300 includes a single server data analysis and storage distribution network, or optionally, a network-based data analysis and storage distribution network 319 in communication with at least one device 306 (and optionally additionally client devices 306, or more). The device 306 may comprise a sensor device (e.g., a spirometer) that is in communication 318 with a data and communications hub 308. Data and communications hub 308 optionally comprises a web client 310 and a programmatic client 312. Exemplary network system 300 may further optionally comprise one or more third-party servers 314 that communicate 318 through cloud communication network 304 with the data and communications hub 308 and/or communicate 318 with the single server data analysis and storage distribution network 319.

The single server data analysis and storage distribution network 319 may be a network-based commercial entity (e.g., a healthcare monitoring and services organization). Device(s) 306 and data and communications hub 308 are representative of a plurality of client device types and are representative of multitude of individual users of the systems and methods of the present disclosure. In one embodiment, device(s) 306 and 308 can be separate devices. In alternative embodiments, the functionalities of the device(s) 306 and hub 308 can be configured into a single multipurpose device that accomplishes the respective functionalities. The present disclosure is not intended to be limited however to one physical configuration of numerated devices, components, systems, or subsystems of the present disclosure. For example, in certain embodiments device(s) 306 comprise two or more devices that monitor or record patient health and wellness information and data including, but not limited to, one or more spirometers, incentive spirometers, fast gas analyzers, accelerometers, pneumotachographs, peak flow meters, or plethysmographs (e.g., photoplethysmographs, optoelectronic plethysmographs, respiratory inductance plethysmographs), inhalation monitors, pulse oximeters, blood pressure meters (e.g., cuffs), blood glucose meters, wearable fitness trackers and monitors, electrocardiogram monitors, thermometers, therapeutic devices and monitors, and the like). In certain embodiments, one or more of these health-related monitoring functionalities can be optionally combined into one, two, three, or more, devices that can be in communication 318 with the hub 308.

The server data analysis and storage distribution network 319 may communicate and exchange data within the network system 300 that may pertain to various functions and aspects associated with the network system 300 and its users, and in particular, user supplied data and health-related information. The server data analysis and storage distribution network 319 may provide server-side functionality, via a cloud communication network 304 (e.g., the Internet), to one or more client devices (e.g., devices 306 and the data and communications hub 308). The one or more devices 306 and hub 308 may be operated by users that use the network system 300 to exchange data over a cloud communication network 304 with the server data analysis and storage distribution network 319 and/or third-party servers 314. These exchanges may include, but are not limited to, transmitting and receiving (i.e., communicating), and processing data regarding personal health-related information sampled or collected by device(s) 306 from the respective user. The data may include, but is not limited to, images, video or audio content, physical information and collected values (e.g., spirometry values), instructions, identifiers, as well de-identifiers and deidentified data, user preferences, averaged data sets, instant data sets, stored and retrieved data sets, among other things.

In various embodiments, the data exchanged within the network system 300 may be dependent upon user-selected functions available through one or more client or user interfaces (UIs) modules. The UIs may be associated with a client device, such as device 306, or alternatively, and in some embodiments, associated with data and communications hub 308 using web a web client 310 and/or a programmatic client 312. In various embodiments, the data and communications hub 308 through programmatic and/or web clients 310/312 may communicate through the cloud communication network 304 with the server data analysis and storage distribution network 319 via a web server 322. The UIs may also be associated with the device 306 through the data and communications hub 308 using a programmatic client 312, such as a client application in communication with the server data analysis and storage distribution network 319, or optionally a third-party server 314 hosting one or more third-party application(s) 316.

In some embodiments the hub 308 can be a purpose-built device for bidirectional communication (and optionally, data analysis, processing, data storage, and/or data display or projection) with the server data analysis and storage distribution network 319 and/or third-party servers 314. The data and communications hub 308 may however alternatively be any one of a variety of types of devices such as, but not limited to: 1) a mobile communications device such, a cellular phone, a smartphone ((e.g., iPhone® (Apple Inc., Cupertino, Calif.), or other mobile device running the iOS® operating system, or the Android® (Google Inc., Mountain View, Calif.) operating system, a BlackBerry® (Blackberry Ltd., Waterloo, Canada) operating system, the Microsoft® Windows® Phone (Microsoft Inc., Redmond, Wash.) operating system, Symbian® OS (Symbian Ltd., London, UK), or WebOS® (LG Corp., Seoul, South Korea)); or 2) a portable networked device; 3) a tablet computer, such as an iPad® or other tablet computer running one of the aforementioned operating systems; 4) a Personal Digital Assistant (PDA); 5) a handheld computer; 6) a desktop computer; 7) a laptop computer or netbook; 8) a wearable computing device such as glasses or a wristwatch; 9) a multimedia device; 10) a data enabled book reader; or 11) a video game system console, such as, the Microsoft Xbox 360®, or the SONY PLAY STATION 3® (Sony Corp., Tokyo, Japan) or other video game system consoles and portable gaming devices. According to one embodiment, the data and communications hub 308 can comprise a standard or “off-the-shelf” device such as a tablet, personal computer, cell phone, or other such device with a client application 310 and/or 312 pre-installed thereon. Such an application can enable functions of the data and communications hub 308 as described herein while, in some cases, disabling or preventing access to other functions of the device and thus turning the device into a dedicated data and communications hub 308.

Client devices 306 and data and communications hub 308 may be arranged to transmit and receive wireless signals via an interface (connection) 318 with the cloud communication network 304 (e.g., the Internet or Wide Area Network (WAN)). Depending on the form of the device 306 and hub 308, any of a variety of types of connections 318 (either persistent or intermittent) and cloud communication networks 304 may be used. For example, the connection 318 may be Code Division Multiple Access (CDMA) connection, a Global System for Mobile communications (GSM) connection, Bluetooth®, Wireless Fidelity (Wi-Fi®; IEEE 802.11x type) cellular, radio-frequency identification (MD), Worldwide Interoperability for Microwave Access (WiMAX®), or other type of wireless data connection. Such a connection 318 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, or other data transfer technology (e.g., fourth generation wireless, 4G networks, fifth generation wireless, 5G networks). When such technology is employed, the cloud communication network 304 may include a cellular network that has a plurality of cell sites of overlapping geographic coverage, interconnected by cellular telephone exchanges. These cellular telephone exchanges may be coupled to a network backbone (e.g., the public switched telephone network (PSTN), a packet-switched data network, or to other types of networks).

In such an embodiment, the cloud communication network 304 may include one or more wireless access points coupled to a local area network (LAN), a WAN, the Internet, or other packet-switched data network. In yet another example, the connection 318 may be a wired connection, for example an Ethernet link, USB, IEEE1394, and the like, and the cloud communication network 304 may be a LAN, a WAN, the Internet, or other packet-switched data network. Accordingly, a variety of different configurations are expressly contemplated.

Turning specifically to the server data analysis and storage distribution network 319, the API server 320 and a web server 322 are coupled to, and provide programmatic and web interfaces respectively to, one or more application servers 324. In some embodiments, server data analysis and storage distribution network 319 can be configured to employ operating principles of the internet of things “IoT”; and more particularly, be configured to operate using an open OASIS and ISO standard (i.e., ISO/IEC 20922) lightweight publish-subscribe network protocol that implements a client/broker architecture such as the Message Queuing Telemetry Transport “MQTT” protocol (e.g., MQTT v3.1-5). It is understood that the MQTT protocol typically runs over TCP/IP or another suitable suite that can provide ordered, lossless, bi-directional connections (e.g., one or more of BGP, DHCP, DNS, FTP, HTTP, HTTPS, IMAP, LDAP, MGCP, NNTP, NTP, POP, PTP, ONC/RPC, RTP, RTSP, RIP, SIP, SMTP, SNMP, SSH, Telnet, TLS/SSL, XMPP, and the like). Consequently, while the systems and methods of the present disclosure are not limited to using the MQTT protocol, as other protocols may find use with the disclosure when they are designed to work in remote locations where a “small code footprint” is required, the communications network has limited bandwidth, or the implementing devices have limited power.

The application servers 324 may, for example, host user specific healthcare monitoring and alert applications 326, which may provide several healthcare, wellness, and emergency response related functions and services to users that access the server data analysis and storage distribution network 319. For example, the application servers 324 can provide any number and variety of reports, textual and/or graphical user interfaces, and/or other presentations of the date collect from the health data sensing device 306 and provided by the data and communications hub 308. In this way, physicians, therapists, and other authorized users can review and monitor the data as part of providing treatment or advise to the user. Additionally, or alternatively, applications executed by the application servers 324 can automatically monitor such data to, for example, detect anomalies therein, make judgements on the user's current health, make predictions about the user's future health, etc.

Accordingly, in some embodiments, the user specific healthcare monitoring and alert applications 326, may be configured to generate data and reports related to the user's health status (e.g., pulmonary function and/or other physiological function(s)) and to present the data and reports to the user and optionally to others as is advantageous to maintain, treat, ameliorate, or further provide for the care and monitoring of at least one aspect of the user's health (e.g., respiratory and pulmonary health) and wellbeing. The application servers 324 may be coupled via these interfaces to the cloud communication network 304, for example, via wired or wireless interfaces. The application servers 324 are, in turn, shown to be coupled to one or more database servers 330 that facilitate access to one or more databases 332. In some examples, the application servers 324 can access the databases 332 directly without the need for a database server 330. In some embodiments, the databases 332 may include or access databases both internal and external to the server data analysis and storage distribution network 319 (e.g., third-party server 314, database 317).

While the user specific healthcare monitoring and alert applications 326 are shown in FIG. 3A to form part of the server data analysis and storage distribution network 319, it will be appreciated that, in alternative embodiments, the user specific healthcare monitoring and alert applications 326 may form part of a service that can be separate and distinct from the server data analysis and storage distribution network 319. Further, while the system 300 shown in FIG. 3A employs client-server architecture, the present inventive subject matter is, of course, not limited to such an architecture, and could equally well find application in an event-driven, distributed, or peer-to-peer architecture system, for example. The various applications and modules of the application servers 324 may also be implemented as standalone systems or software programs, which do not necessarily have networking capabilities.

FIG. 3A also further illustrates an optional third-party application(s) 316 executing on the third-party server(s) 314 that may offer one or more services to users of the device 306 and hub 308. In one exemplary embodiment, the third-party application 316 comprises an analysis function for the data and information transmitted to it by device 306 user via data and communication hub 308. More particularly, the user transmitted data and information can be evaluated by third-party application 316 to determine at least one aspect of the present health status of the user, or optionally, a trend in user's health, and/or still optionally, to enter (e.g., recordation in a database) the user's transmitted data and information for subsequent processing. In one exemplary illustration of a contemplated embodiment, the third-party application 316 receives the user's transmitted data and information and analyzes it in real-time to send an alarm, notice, or instructions back to the user and/or to a subsequent third-party server(s) 314. Third-party application 316 can make use of human analysts (e.g., data analysts), clinicians and healthcare providers (e.g., doctors, nurses, counselors, first responders, and the like), caregivers, and insurance providers as well as additional computer implemented applications such as Artificial Intelligence (AI) applications, neural networks, and systems.

The third-party application 316 may have programmatic access to the server data analysis and storage distribution network 319 via the programmatic interface provided by an application program interface (API) server 320. In some embodiments, a third-party application 316 may be associated with third-party organizations (e.g., medical practices, hospitals, managed care organizations, insurance providers) and other merchants. Third-party application 316 may be optionally communicate with digital wallets functionalities, payment services (e.g., Apple Pay®, Google Wallet® (Google Inc., Mountain View, Calif.), Stripe® (Stripe Inc., San Francisco, Calif.), Square® (Square Inc., San Francisco, Calif.), and Payza® (Payza Ltd., London, UK) and the like) or any organizations that may conduct transactions with or provide services (e.g., social media tools) to the users of the device(s) 306 and data and communications hub 308.

FIG. 3B is a block diagram illustrating an alternative exemplary environment in which embodiments of the present disclosure may be implemented. This example illustrates the system 300 described above with reference to FIG. 3A. However, as shown in FIG. 3B, device 306 can optionally comprises a web client 313.

Stated another way, monitoring telemetric health data can comprise receiving, by a server 314 or 319 of a telemetric health data monitoring system 300, set-up data for a telemetric health data sensing device 306, and maintaining, by the server 314 or 319 of a telemetric health data monitoring system 300, the received set-up data for the telemetric health data sensing device 306 in an electronic record, i.e., in a database 317 or 332, corresponding to a user of the telemetric health data sensing device 306. The set-up data for the telemetric health data sensing device 306 can comprise one or more of a serial number for the telemetric health data sensing device, a date, or demographic information for the user of the telemetric health data sensing device.

A determination can be made by the server 314 or 319 of the telemetric health data monitoring system 300 as to whether to collect telemetric health data corresponding to the user of the telemetric health data sensing device 306. Determining whether to collect the telemetric health data corresponding to the user of the telemetric health data sensing device 306 can be based at least in part on the set-up data for the telemetric health data sensing device. In response to determining to collect the telemetric health data corresponding to the user of the telemetric health data sensing device 306, an electronic message requesting the telemetric health data can be sent by the server 314 or 319 of the telemetric health data monitoring system 300 via a communications network 304 to a hub device 308 communicatively coupled with the telemetric health data sensing device 306. The electronic message requesting the telemetric health data can comprise one or more of instructions to the hub device 308 or instructions to the user of the telemetric health data sensing device 306.

The hub device 308 of the telemetric health data monitoring system 300 can receive, from the server 314 or 319 of the telemetric health monitoring system 300, via the communications network 304, the electronic message requesting telemetric health data corresponding to a user of a telemetric health data sensing device 306, collect data from the user of the telemetric health data sensing device 306 and/or collect the telemetric health data from the telemetric health data sensing device 306. The requested telemetric health data can comprise, for example, one or more of a serial number for the telemetric health data sensing device 306, a time stamp for the requested telemetric health data, one or more data labels and numerical values for the requested telemetric health data, or questionnaire answers from the user of the telemetric health data sensing device 306. In some cases, collecting data from the user of the telemetric health data sensing device 306 can further comprise waking up the telemetric health data sensing device 306. The data collected from the user of the telemetric health data sensing device 306 and the telemetric health data collected from the telemetric health data sensing device 306 can then be provided by the hub device 308 of the telemetric health data monitoring system 300 to the server 314 or 319 via the communications network 304 in response to the received electronic message.

The server 314 or 319 of the telemetric health data monitoring system 300 can receive the data collected from the user of the telemetric health data sensing device 306 and the telemetric health data collected from the telemetric health data sensing device 306 from the hub device 308 via the communications network 304 and update the electronic record corresponding to the user of the telemetric health data sensing device 306 with the received the data collected from the user of the telemetric health data sensing device 306 and the telemetric health data collected from the telemetric health data sensing device 306.

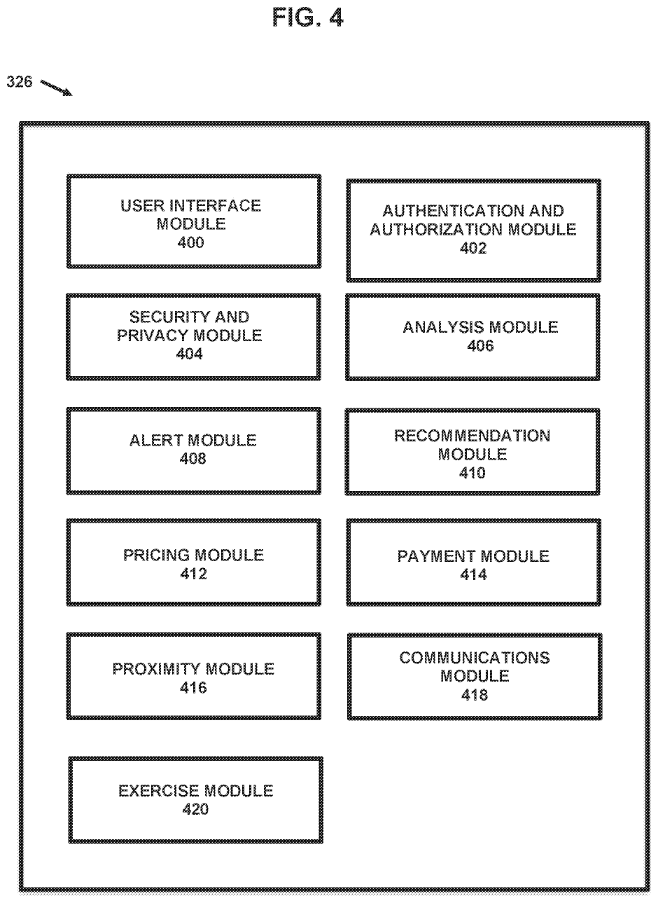

FIG. 4 is a block diagram illustrating an exemplary embodiment of various modules forming a user specific healthcare monitoring and alert applications, which can be provided as part of the network system of FIG. 3A and FIG. 3B. The modules of the user specific healthcare monitoring and alert applications 426 may be hosted on dedicated or shared server machines that can be communicatively coupled to enable communications between server machines. Each of the modules 400-418 can be communicatively coupled (e.g., via appropriate interfaces) to each other and to various data sources, to allow information to be passed between the modules 400-418 of the user specific healthcare monitoring and alert applications 426 or to allow the modules 400-418 to share and access common data. The modules 400-418 may optionally be communicatively coupled to enable communications and information to flow between one or more modules 400-418 and devices 306 and/or data and communications hub 308 (e.g., an embedded device) through interface (connection) 418 with the cloud communication network 304. The various modules of the user specific healthcare monitoring and alert applications 426 may furthermore access one or more databases 332 via the database servers 330. Further, while the modules of FIG. 4 are discussed in the singular sense, it will be appreciated that in other embodiments multiple modules may be employed.

The user specific healthcare monitoring and alert applications 426 may provide a number of analysis, authentication, security and privacy, alert, storage, and the like whereby the system user interacts (e.g., provides data and information and received data and information) with concerning their health status (e.g., respiratory and pulmonary health) and wellbeing such that a user can interact with server data analysis and storage distribution network 3419 to make healthcare related decisions and the like. To this end, the user specific healthcare monitoring and alert applications 426 are shown to include at least one user interface module 400 that can be configured to receive data and information acquired from device 306 that was transmitted via the data and communications hub 308 via cloud communications network 304. The user interface module 400 can be similarly configured to transmit data and information to the user through the data and communications hub 308 via cloud communications network 304. Additionally, applications 426 can be configured through user interface module 400, or otherwise, to transmit data and information to the one or more third-party servers 314.

Also, in certain embodiments can be an authentication and authorization module 402 that can be configured to verify the validity and veracity of the data and information from the user received from as well as transmitted to the system user. While the present invention is not intended to be limited to any particular number or sequence of verifications provide by authentication and authorization module 402, the present invention contemplates that verification include, at least, those commonly provided in identity and access management “IAM” routines, including, but not limited to: determining whether users are who they claim to be; optionally challenging users to validate credentials (e.g., through passwords, answers to security questions, or facial recognition, and the like); optionally administering ID token and/or access token systems; determining what users can and cannot access; verifying whether access is allowed through policies and rules; and the like.

Similarly, user specific healthcare monitoring and alert applications 426, can comprise a security and privacy module 404 that can be configured to comprises assurance steps for compliance with applicable economic union, federal, state, provincial, and local, data privacy laws and regulations (e.g., HIPPA, GDPR, and the like) concerning potentially sensitive healthcare related data and information. In addition providing data privacy assurances, this module (404) can optionally further be configured to provide one or more commonly understood principles and components of cyber security such as, but not limited to: governing (e.g., identifying and managing security risks); protecting (e.g., implementing security controls to mitigate/reduce security risks); detecting (e.g., detecting and understanding malicious security events); and responding (e.g., responding to and recovering from security incidents) as these principles are understood by those skilled in the art to relate to unauthorized attempted incursions, breaches, and corruption of data stored in database 332.

In some embodiments, the user data and information received by the user specific healthcare monitoring and alert applications 426 can be manipulated, compared, and interpreted in the analysis module 406. More particularly, analysis module 406 is contemplated to resolve the user's inputs for deviation from pooled averages (or accumulated from the user) for the types of data measured by device(s) 306, and/or deviation from established trends for the user, and/or values that are known to be indicative of health consequences (e.g., a worsening or an improving health status), among of potential analyses understood as applicable by those skilled in the art. Certain pieces of user provided data and information may be deidentified as required to compliance with applicable data privacy and confidentiality regimes.

Additional embodiments user specific healthcare monitoring and alert applications 426 optionally comprise an alert module 408 configured to contemporaneously notify one or more of: a user; a first responder proximate to the user; and/or an emergency medicine provider proximate to the user of a potentially dire and impending health emergency.

As illustrated additionally in FIG. 4, the user specific healthcare monitoring and alert applications 426 therein may include one or more recommendation modules 410 that can be configured to provide recommendations to the user for obtaining services, products, user gatherings (e.g., social gatherings, exercise classes, support groups, and the like) that the user might want to consider as they relate to improving or to maintain their health and wellbeing. Additionally, module 410 can be configured to provide the user with generalized (or tailored and specific) healthcare and wellbeing information of interest as indicated, or intuited by the system, from user inputs. Embodiments of applications 426 comprising one or more recommendation modules 410 can provide geolocated information, services, products, and offerings that the user can source or obtain within a preset radius (e.g., within 0.25, 0.50, 1, 2, 5, 10, 100, or more, kilometers or miles) of the user's domicile or their location when using the systems and methods of the present invention. The radius can be specified by the user's stated preferences (inputs) or is intuited by the system from patterns from that user's or other users' observed preferences and other inputs. In some embodiments, the recommendation module 410 may receive requests for recommendations, from the user (and/or users) and in turn provide them with a recommendation based on information contained in the user's, seller's, service provider's corresponding profile(s) including available product inventories and other parameters. In some embodiments, the recommendation module 410 may automatically generate and transmit a recommendation (e.g., a push notification) to a receptive user based on the user's proximity to an anticipated point or item of potential interest according to their previous interactions with the system. The recommendations provided by the recommendation module 410 may contain one or more items (e.g., goods or services offered) that may potentially interest a user. In various embodiments comprising one or more recommendation modules 410, the user specific healthcare monitoring and alert applications 426 optionally further comprises one or both of a pricing module 412 and optionally a payment module 414.

The communications module 418 may utilize any one of several message delivery networks and platforms to deliver messages to users. For example, the communications module 418 may deliver push notifications via a suitable push notification service (e.g., electronic mail (e-mail), instant message “IM,” Short Message Service “SMS,” text, facsimile, or voice (e.g., voice over IP “VoIP”) messages via the wired (e.g., the Internet), plain old telephone service “POTS,” or wireless (e.g., mobile, cellular, WiFi, WiMAX)).

As further illustrated in FIG. 4, the user specific healthcare monitoring and alert applications 426 may include one or more proximity module(s) 416. The proximity module 416 may periodically, continuously, or regularly obtain a user's location. In one embodiment, the user's location can be determined with technologies such as Global Positioning System (GPS), assisted GPS, radio frequency triangulation (e.g., cellular triangulation or Wi-Fi triangulation), or other location aware services. The proximity module 416 may communicate with a server (e.g., a cloud-based service) to obtain the location of device 306 and the data and communications hub 308. The device 306 and data and communications hub 308 may be configured to transmit and receive wireless signals using various technologies. Examples of wireless technologies include, but are not limited to, Bluetooth® (Bluetooth Special Interest Group, Kirkland, Wash.), Wi-Fi, cellular, radio-frequency identification, WiMAX, and the like.

In still other embodiments, user specific healthcare monitoring and alert applications 426 additionally comprises an exercise module 420 that can be configured to receive and provide exercise and/or exercise physiology related data from a user as well as optionally push exercise related directions, alerts (e.g., target heart rate, heartbeat, respiratory, pulse oximetry, and the like, monitoring alerts), and instructions (e.g., exercise programs, hydration reminders and the like) to the user. In embodiments optionally comprising an exercise module 420, the system can be further configured to operate additional user data testing and monitoring devices, inputs, and outputs.

It is to be understood that any one or more of modules 400-418, as optionally provided in application 426, may be provisioned by third parties.

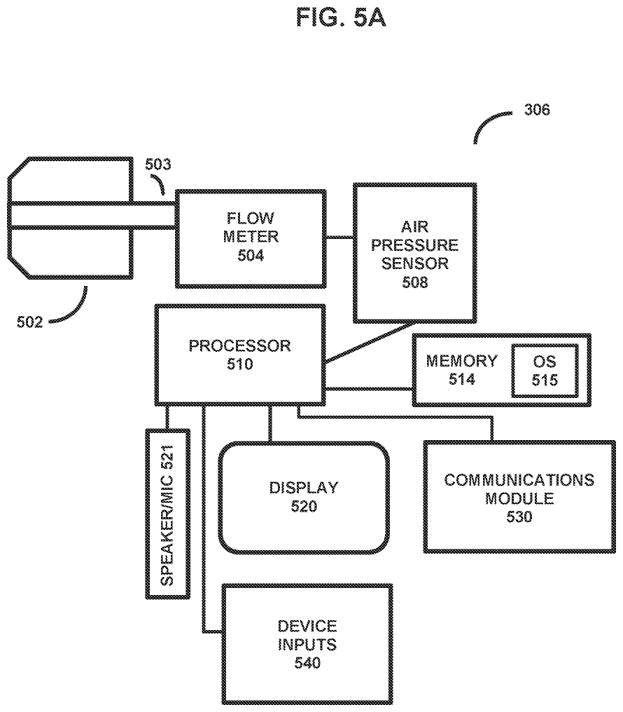

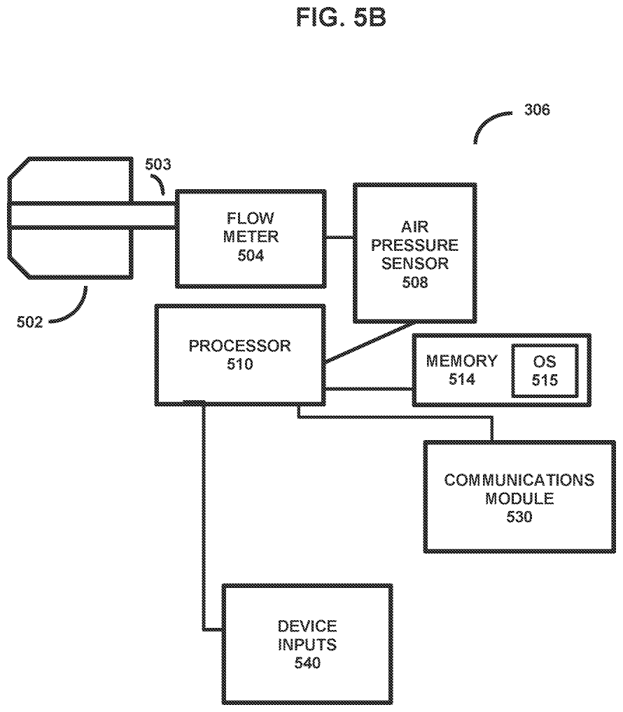

FIGS. 5A and 5B are block diagrams of exemplary devices and components therein according to embodiments of the present disclosure. More specifically, FIG. 5A is a block diagram illustrating a device 306, and more particularly, an embodiment where device 306 can be a portable spirometer 500 consistent with some embodiments. Spirometer device 306 comprises an air channel 502 which has a center channel 503 for receiving the exhalation and/or inhalation of the user. In various embodiments, channel 503 leads to a flow meter 504 which operates to create a pressure difference, in a manner known in the art, to provide an input to air pressure sensor 508. The air pressure sensor 508 compares the pressure induced by the respired air to the ambient air pressure and outputs an electrical current output typically between 1-5 Hz to a further embedded device and single processing component such as a conditioning amplifier, an analog to digital converter and the like. In one such contemplated embodiment, the conditioning amplifier optionally amplifies the output signals of the air pressure sensor 508 to a level accepted by convertor, such as, but not limited to, an analog to digital converter “A/D” in certain embodiments. Via A/D converter, the conditioning amplifier outputs a digital signal (e.g., at a sampling rate of typically about 400 cycles per second) to processor 510 (e.g., central processor “CPU”) which may be any suitably featured and powerful processor. The processor 510 may be any of a variety of different types of commercially available processors suitable for mobile devices (e.g., an XScale architecture microprocessor, a Microprocessor without Interlocked Pipeline Stages “MIPS” architecture processor, or another type of processor). In various embodiments, processor 510 optionally includes a Graphical User Interface “GUI.” The processor 510 may be further coupled, as shown below, either directly or via appropriate intermediary hardware and software, to one or more input/output “I/O” devices, such as display 520, speaker/microphone 521, device inputs 540, and a camera. Similarly, in some embodiments, the processor 510 may be coupled to a transceiver that interfaces with an antenna.

Processor 510 can be operably linked to memory 514 and configured to receive and store in a retrievable and at least semi-permanent manner input data and other system controls and operating routines that can be in turn executed by processor 510. The flow of information between processor 510 and memory 514 is contemplated to be dynamic and bidirectional.

Memory 514 is typically a random-access memory “RAM,” a Flash memory, or other type of memory, that is accessible to the processor 510. The memory 514 may store instructions for execution by the processor 510 for causing the device 306 to perform specific tasks. For example, the memory 514 may be adapted to store an operating system “OS” (515).

As illustrated in FIG. 5A, processor 510 can be configured to output user perceptible information such as, for example, instructions for operating device 306, operational status of device 306 (e.g., existing internal power (e.g., battery) levels; current input mode status; current output transmitting mode status; indications of communications operability and strength status (e.g., communications with data and communications hub 308); operational codes; and other information and health assessments). In favored embodiments, the processor 510 outputs user perceptible information to display 520 when the information can be expressed in a visually perceptible medium, and to the speaker (and optional microphone) 521 when the information is expressed as sound. In various embodiments, speaker 521 combines microphone functionality thus allowing the user to input information to device 306 through speech; in these embodiments, processor 510 can be configured to receive and decode spoken instructions from the user. It is further contemplated that device 306, in favored embodiments, can be configurable to interface (i.e., receive and deliver instructions and information) with the user through display 520, speaker/microphone 521, or a combination of both. Device 306 may optionally employ haptic sensors and stimulus generators in various other embodiments.

In addition to the user's capability to input and receive instructions and information from device 306 through display 520 and speaker/microphone 521, in other embodiments, the device 306 further comprises device inputs 540. It will be understood that device input(s) 540 may comprise, but not be limited to, one or more buttons (e.g., power on/off/standby), switches, dials, sliders, and the like, light or infra-red sensors, keyboards, and pressure or conductance sensitive display(s).

FIG. 5A further illustrates device 306 comprising a communications module 530. In various embodiments, communications module 530 receives information and data from processor 510 and relays (transmits) the information and data to a data and communications hub 308 in relative proximate thereto.

The communications module 530 may be configured to function as a transceiver to both transmit and receive signals (e.g., wireless data signals), or other types of signals via an antenna, depending on the nature of the location device 306. In this manner, communication 318 with the cloud communication network 304 may be established through the data and communications hub 308 intermediary. While various communications schemes can be employed between communications module 530 and the data and communications hub 308, it is contemplated, as described above, in relation to communications with the single server data analysis and storage distribution network 319, that the communications be facilitated using a wireless lightweight publish-subscribe network protocol that implements a client/broker architecture such as the MQTT protocol (e.g., MQTT v3.1-5).

Device 306 may also be configured to determine its location and/or the location for data and communications 308 by using an internet protocol “IP” address lookup or by triangulating a position based on nearby mobile communications towers. In these embodiments, the optional location feature may be further configured to store a user-defined location in memory 514. In some embodiments, a location enabled application may work in conjunction with the location feature and the communications module 530 to transmit the location of device 306 and/or data and communications hub 308 to the single server data analysis and storage distribution network 319 and/or third-party server 314.

According to one embodiment, the device 306 may be provided without a battery or other internal power supply. In such cases, the device 306 may be powered only when connected with the data and communications hub device 308. For example, the health data sensing device 306 can be coupled with the data and communications hub device 308 via a wired connections such as a USB port and cable, for example. Thus, power can be provided to heath data sensing device 308 from the data and communications hub device 308 and can be started or awoken once the data and communications hub device 308 detects the connection with the health data sensing device 306. In this way, security of the device 306 can be improved since it is only powered when connected with the data and communications hub device 308 and encryption and security features can be enforced by the data and communications hub device 308.

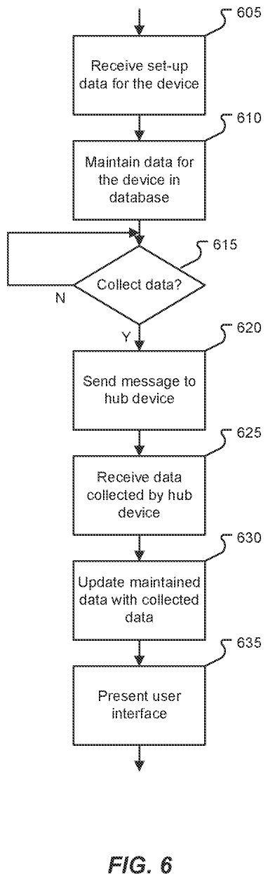

FIG. 6 is a flowchart illustrating an exemplary process for telemetric health monitoring according to one embodiment of the present disclosure. More specifically, this example illustrates processes as may be performed by a server 314 or 319 of a telemetric health data monitoring system 300 as described above. As illustrated in this example, monitoring telemetric health data can comprise receiving 605 set-up data for a telemetric health data sensing device and maintaining 610 the received set-up data for the telemetric health data sensing device in an electronic record corresponding to a user of the telemetric health data sensing device. For example, the set-up data for the telemetric health data sensing device can comprise one or more of a serial number for the telemetric health data sensing device, a date, or demographic information for the user of the telemetric health data sensing device.

A determination 615 can then be made as to whether to collect telemetric health data corresponding to the user of the telemetric health data sensing device. For example, determining 615 whether to collect the telemetric health data corresponding to the user of the telemetric health data sensing device can be based at least in part on the set-up data for the telemetric health data sensing device, e.g. by applying rules, a learned model etc., to the demographic data for the user of the device.

In response to determining 615 to collect the telemetric health data corresponding to the user of the telemetric health data sensing device, an electronic message requesting the telemetric health data to a hub device communicatively coupled with the telemetric health data sensing device can be sent 620 to a hub device 308 as described above. In reply, the requested telemetric health data can be received 625 from the hub device via the communications network, and the electronic record corresponding to the user of the telemetric health data sensing device can be updated 630 with the received telemetric health data. In some cases, the electronic message requesting the telemetric health data can comprise one or more of instructions to the hub device or instructions to the user of the telemetric health data sensing device. The requested telemetric health data can comprise, for example, one or more of a serial number for the telemetric health data sensing device, a time stamp for the requested telemetric health data, one or more data labels and numerical values for the requested telemetric health data, or questionnaire answers from the user of the telemetric health data sensing device. In some cases, a graphical user interface presenting at least a portion of the telemetric health data maintained in the electronic record corresponding to the user of the telemetric health data sensing device can be provided 635.

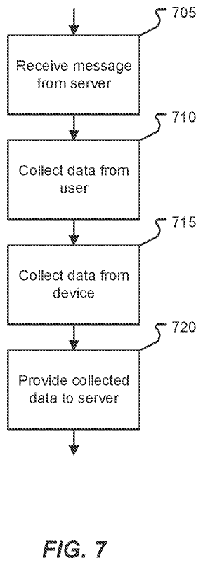

FIG. 7 is a flowchart illustrating an exemplary process for telemetric health monitoring according to another embodiment of the present disclosure. More specifically, this example illustrates processes as may be performed by a data and communications hub 308 as described above. As illustrated here, monitoring telemetric health data can comprise receiving 705 an electronic message requesting telemetric health data corresponding to a user of a telemetric health data sensing device. In some cases, the electronic message requesting telemetric health data corresponding to the user of a telemetric health data sensing device further can comprise instructions to the user of the telemetric health data sensing device.

The telemetric health data can be collected 710 from the user and collected 715 from the telemetric health data sensing device. The telemetric health data collected 715 from the telemetric health data sensing device can comprise one or more of a serial number for the telemetric health data sensing device, a time stamp for the telemetric health data, or one or more data labels and numerical values for the telemetric health data. In some cases, collecting 710 data from the user of the telemetric health data sensing device and/or collecting 715 telemetric health data from the telemetric health data sensing device can further comprise providing a wake up instruction to the telemetric health data sensing device. Additionally, or alternatively, collecting 710 data from the user of the telemetric health data sensing device can comprise presenting a questionnaire to the user of the telemetric health data sensing device and wherein the data collected from the user of the telemetric health data sensing device comprises answers to the questionnaire. The data collected 710 from the user of the telemetric health data sensing device and the telemetric health data collected 715 from the telemetric health data sensing device can then be provided 720 to the server via the communications network in response to the received electronic message.

The present disclosure, in various aspects, embodiments, and/or configurations, includes components, methods, processes, systems, and/or apparatus substantially as depicted and described herein, including various aspects, embodiments, configurations embodiments, sub-combinations, and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and\or reducing cost of implementation.

The foregoing discussion has been presented for purposes of illustration and description. The foregoing is not intended to limit the disclosure to the form or forms disclosed herein. In the foregoing Detailed Description for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A server of a telemetric health monitoring system, the server comprising:
a processor; and
a memory coupled with and readable by the processor and storing therein a set of instructions which, when executed by the processor, causes the processor to:
receive set-up data for a telemetric health data sensing device,
maintain the received set-up data for the telemetric health data sensing device in an electronic record corresponding to a user of the telemetric health data sensing device,
determine whether to collect telemetric health data corresponding to the user of the telemetric health data sensing device, and
in response to determining to collect the telemetric health data corresponding to the user of the telemetric health data sensing device, sending an electronic message requesting the telemetric health data to a hub device communicatively coupled with the telemetric health data sensing device via a communications network, receiving the requested telemetric health data from the hub device via the communications network, and updating the electronic record corresponding to the user of the telemetric health data sensing device with the received telemetric health data.

2. The server of claim 1, wherein the set-up data for the telemetric health data sensing device comprises one or more of a serial number for the telemetric health data sensing device, a date, or demographic information for the user of the telemetric health data sensing device.

3. The server of claim 1, wherein determining whether to collect the telemetric health data corresponding to the user of the telemetric health data sensing device is based at least in part on the set-up data for the telemetric health data sensing device.

4. The server of claim 1, wherein the electronic message requesting the telemetric health data comprises one or more of instructions to the hub device or instructions to the user of the telemetric health data sensing device.

5. The server of claim 1, wherein the requested telemetric health data comprises one or more of a serial number for the telemetric health data sensing device, a time stamp for the requested telemetric health data, one or more data labels and numerical values for the requested telemetric health data, or questionnaire answers from the user of the telemetric health data sensing device.

6. The server of claim 1, wherein the instructions further cause the processor to present a graphical user interface presenting at least a portion of the telemetric health data maintained in the electronic record corresponding to the user of the telemetric health data sensing device.

7. A data and communications hub device of a telemetric health monitoring system, the data and communications hub comprising:
a processor; and
a memory coupled with and readable by the processor and storing therein a set of instructions which, when executed by the processor, causes the processor to:
receive, from a server of the telemetric health monitoring system, via a communications network, an electronic message requesting telemetric health data corresponding to a user of a telemetric health data sensing device,
collect data from the user of the telemetric health data sensing device,
collect the telemetric health data from the telemetric health data sensing device, and
provide the data collected from the user of the telemetric health data sensing device and the telemetric health data collected from the telemetric health data sensing device to the server via the communications network in response to the received electronic message.

8. The data and communications hub device of claim 7, wherein the electronic message requesting telemetric health data corresponding to the user of a telemetric health data sensing device further comprises instructions to the user of the telemetric health data sensing device.

9. The data and communications hub device of claim 7, wherein the telemetric health data collected from the telemetric health data sensing device comprises one or more of a serial number for the telemetric health data sensing device, a time stamp for the telemetric health data, or one or more data labels and numerical values for the telemetric health data.

10. The data and communications hub device of claim 7, wherein collecting data from the user of the telemetric health data sensing device further comprises providing a wake up instruction to the telemetric health data sensing device.

11. The data and communications hub device of claim 7, wherein collecting data from the user of the telemetric health data sensing device further comprises presenting a questionnaire to the user of the telemetric health data sensing device and wherein the data collected from the user of the telemetric health data sensing device comprises answers to the questionnaire.

12. A method for monitoring telemetric health data, the method comprising:
receiving, by a server of a telemetric health data monitoring system, set-up data for a telemetric health data sensing device;
maintaining, by the server of a telemetric health data monitoring system, the received set-up data for the telemetric health data sensing device in an electronic record corresponding to a user of the telemetric health data sensing device;
determining, by the server of the telemetric health data monitoring system, whether to collect telemetric health data corresponding to the user of the telemetric health data sensing device;
in response to determining to collect the telemetric health data corresponding to the user of the telemetric health data sensing device, sending, by the server of the telemetric health data monitoring system, an electronic message requesting the telemetric health data to a hub device communicatively coupled with the telemetric health data sensing device via a communications network.

13. The method of claim 12, further comprising:
receiving, by the hub device of the telemetric health data monitoring system, from the server of the telemetric health monitoring system, via the communications network, the electronic message requesting telemetric health data corresponding to a user of a telemetric health data sensing device;
collecting, by the hub device of the telemetric health data monitoring system, data from the user of the telemetric health data sensing device;
collecting, by the hub device of the telemetric health data monitoring system, the telemetric health data from the telemetric health data sensing device; and
providing, by the hub device of the telemetric health data monitoring system, the data collected from the user of the telemetric health data sensing device and the telemetric health data collected from the telemetric health data sensing device to the server via the communications network in response to the received electronic message.

14. The method of claim 13, further comprising:
receiving, by the server of the telemetric health data monitoring system, the data collected from the user of the telemetric health data sensing device and the telemetric health data collected from the telemetric health data sensing device from the hub device via the communications network; and
updating, by the server of the telemetric health data monitoring system, the electronic record corresponding to the user of the telemetric health data sensing device with the received the data collected from the user of the telemetric health data sensing device and the telemetric health data collected from the telemetric health data sensing device.

15. The method of claim 13, wherein the set-up data for the telemetric health data sensing device comprises one or more of a serial number for the telemetric health data sensing device, a date, or demographic information for the user of the telemetric health data sensing device.

16. The method of claim 13, wherein determining whether to collect the telemetric health data corresponding to the user of the telemetric health data sensing device is based at least in part on the set-up data for the telemetric health data sensing device.

17. The method of claim 13, wherein the electronic message requesting the telemetric health data comprises instructions to the hub device.

18. The method of claim 13, wherein the requested telemetric health data comprises one or more of a serial number for the telemetric health data sensing device, a time stamp for the requested telemetric health data, one or more data labels and numerical values for the requested telemetric health data, or questionnaire answers from the user of the telemetric health data sensing device.

19. The method of claim 13, wherein the electronic message requesting telemetric health data corresponding to the user of a telemetric health data sensing device further comprises instructions to the user of the telemetric health data sensing device.

20. The method of claim 13, wherein collecting data from the user of the telemetric health data sensing device further comprises waking up the telemetric health data sensing device.
